(19) United States
(12) Patent Application Publication
(10) Pub. No.: US 2015/0328395 A1
(43) Pub. Date: Nov. 19, 2015

(54) CLEANING A PLURALITY OF NEEDLES, POST MANUFACTURE AND BEFORE ASSEMBLY INTO A SYRINGE, CANNULA AND THE LIKE

(71) Applicant: JCS-Echigo Pte Ltd, Singapore (SG)

(72) Inventor: Liang Zhao, Singapore (SG)

(21) Appl. No.: 14/405,272

(22) PCT Filed: May 31, 2013
(86) PCT No.: PCT/SG2013/000225

§ 371 Date: Dec. 3, 2014

(30) Foreign Application Priority Data
Jun. 5, 2012 (SG) 201204179-4

(51) Int. Cl. A61M 5/00; B21G 1/12; C25F 3/16; B08B 3/12; B08B 7/04; B08B 11/02

(52) U.S. Cl. CPC A61M 5/001; B08B 7/04; B08B 11/02; C25F 3/16; B08B 3/123; B21G 1/12

(57) ABSTRACT

A system for cleaning a plurality of needles includes a conveyor for conveying a plurality of jigs through a series of stations, and each jig is arranged to contain at least some of the needles. The stations include a pre-treatment station for the removal of detritus from the needles, an electronic polishing station, a post-treatment station for removal of material from the surface of the treated needles, a cleaning station for cleaning the needles, a drying station, and an unloading station for unloading the needles from the jigs.

### REFERENCE TO RELATED APPLICATION

This application is a United States National Phase application of PCT Application No. PCT/SG2013/000225 filed on May 31, 2013, which claims priority to Singapore Application No. 201204179-4 filed on Jun. 5, 2012.

### FIELD OF THE INVENTION

The invention relates to the cleaning of articles, in particular, needles following their manufacture and prior to their assembly.

### BACKGROUND OF THE INVENTION

Following the manufacture of a needle, but prior to the assembly into a subsequent syringe, parenteral cannula devices, or similar the needles, require cleaning. The cleaning may include the removal of burrs and grease as a result of the manufacturing process or other materials, such as cutting fluid etc. The current process involves collecting a plurality of the needles, such as around 10,000, and wrapping the needles in an elastic band to undergo the cleaning process.

The elastic band is intended to prevent the separation of individual needles so that the cleaning process can be applied to the needles on en masse.

However, the application of an elastic band does not provide a controlled application of compression. Accordingly, cleaning the needles is prevented if the elastic band holds the plurality of needles too tightly and if held too loosely, cannot prevent separation/floating of individual needles.

Further, without a controlled compression of the needles preventing the retention of moisture between the needles during the cleaning process becomes more difficult.

For the overall process, the present processes to wash the needles involve a pre-treatment process prior to electro-polishing followed by a post-treatment prior to detergent washing. The washed and polished needles are then passed through several rinsing tanks before being blown dry prior to oven drying and packaging. The process therefore involves significant infrastructure as well as an extended processing time.

### SUMMARY OF INVENTION

In a first aspect the invention provides a system for cleaning a plurality of needles, the system including a conveyor for conveying a plurality of jigs through a series of stations, each jig arranged to contain at least some of the needles. The stations include a pre-treatment station for the removal of detritus from the needles, an electronic polishing station, a post-treatment station for removal of material from the surface of the treated needles, a cleaning station for cleaning the needles, a drying station and, and an unloading station for unloading the needles from the jigs.

In a second aspect the invention provides a method of cleaning a plurality of needles, the method including the steps of conveying a plurality of jigs through a series of stations, each jig arranged to contain at least some of the needles, pre-treating the needles for the removal of detritus, electro-polishing the needles, post-treating the needles to remove material from the surface of the treated needles, cleaning the needles, and drying the needles.

Accordingly, in one aspect, the needles are held in a jig sized to contain a predetermined amount of needles. The tolerance of the jig is such that a number greater or less than the predetermined number will adversely affect the controlled compression, but a balance between “washability” and containment provided.

Further, the needles may be physically restrained within the jig. In one embodiment, a lid is applied to the jig, which may be selectively applied or removed. In a further embodiment, the lid may be an interconnecting section such that the jig and the section are engaged in a complimentary inverted arrangement.

### BRIEF DESCRIPTION OF DRAWINGS

It will be convenient to further describe the present invention with respect to the accompanying drawings that illustrate possible arrangements of the invention. Other arrangements of the invention are possible and consequently, the particularity of the accompanying drawings is not to be understood as superseding the generality of the preceding description of the invention.

FIG. 1 is a flow chart of a process for the cleaning of articles according to one embodiment of the present invention;

FIG. 2 is an elevation view of a needle cleaning device according to one embodiment of the present invention;

FIG. 3 is a plan view of the needle cleaning device of FIG. 2.

FIG. 4A to 4C are isometric views of a basket and jig for containing needles according to a further embodiment of the present invention.

### DETAILED DESCRIPTION

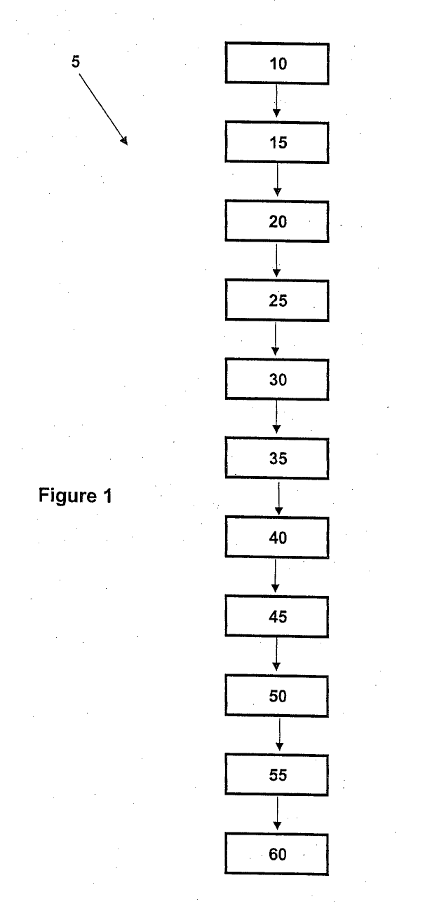

FIG. 1 shows a flow chart of a process for the cleaning of manufactured needles according to one embodiment of the present invention. Here, the process 5 commences with a pre-treatment cycle, such as a high pressure jet spray 10 and/or wash 15 of the needles prior to treatment. The purpose of the pre-treatment is to ensure that when the needles are presented into the electro-polishing 20, detritus does not contaminate the electrolyte solution used for the electro-polishing as well as ensuring an effective electrolysis process in which foreign particles may interfere.

Following the electro-polishing 20, the needles undergo a post-treatment 25, such as emersion into a nitrite acid bath, to remove metal particles from the surface and other residue from the electro-polishing process.

Next, having undergone a rinse and blow-dry, the needles are placed in a washing station, which may go through a path of an ultrasonic pre-wash 30 prior to an ultrasonic detergent wash 35. The needles then undergo a first ultrasonic rinse 40 and if necessary a second ultrasonic rinse 45, with the second ultrasonic rinse being at an elevated temperature, say 90° C.

The needles then undergo a drying sequence of a blow-dry 50 followed by an oven-dry 55 and finishing with a vacuum oven 60 so as to fully remove moisture from the needles. The drying process is facilitated by elevating the rinse water 45 such that any moisture still trapped within the bundle of needles following the blow dry is at an elevated temperature and so the ovens and vacuum oven can more efficiently dry the needles because of the reduced differential temperature of the water as compared to the evaporation temperature.

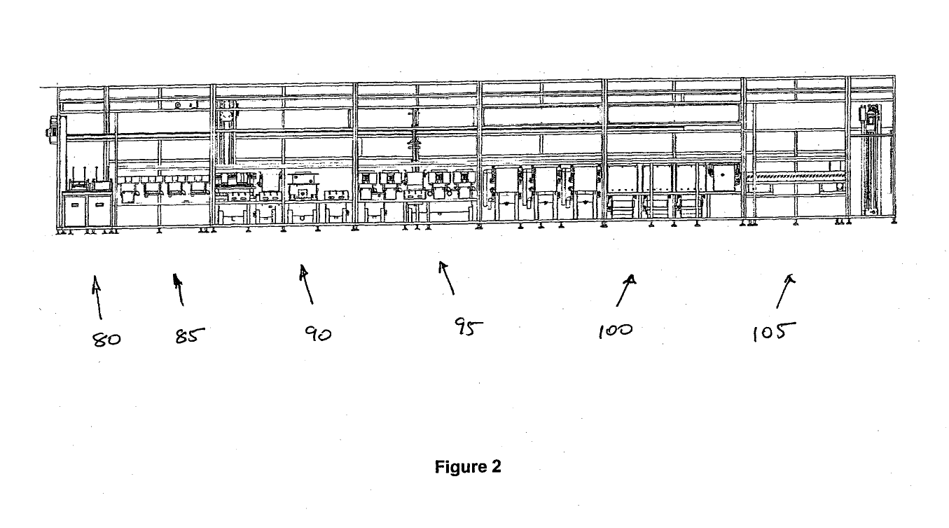

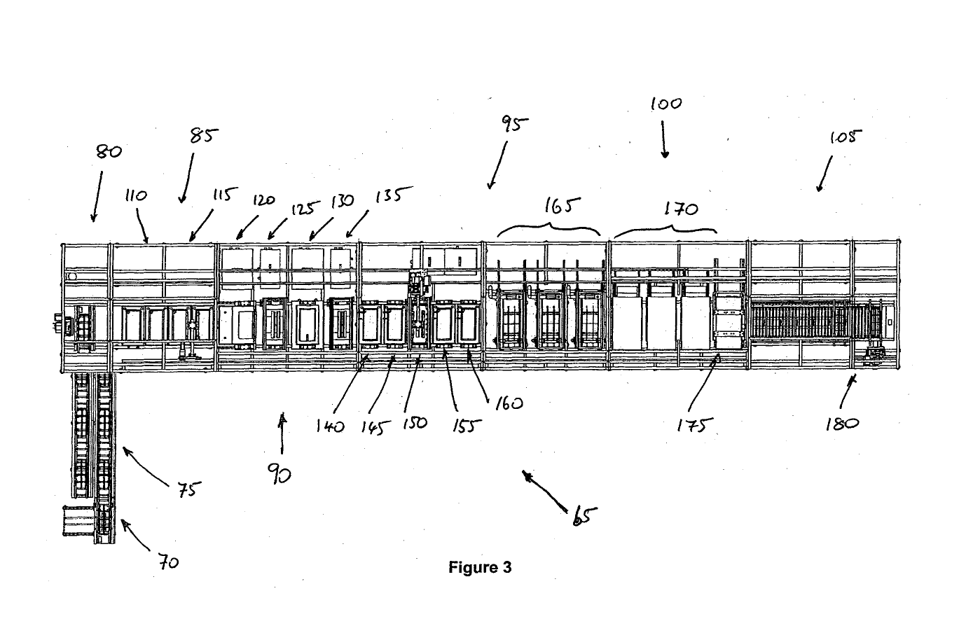

FIGS. 2 and 3 show a plan view and elevation view of a system embodying the method of the present invention. The system 65 is divided into stations commencing with a loading station 80, which delivers the bundle of needles to a pre-treatment station 85, followed by a treatment station 90. The needles then undergo a clean 95 prior to a drying station 100 and finally an unloading station 105.

In particular, the current system includes a conveyor for loading baskets having the jigs placed therein. In order to save on the footprint of the device within the factory environment, the conveyor is placed at right angles to the direction of flow of the baskets through the process. The conveyor receives the baskets at a loading point and delivers the baskets at a first end of the device at the loading station 80. Further, the length of the conveyor provides a buffer to supplement the rate which baskets are loaded into the device and the rate which they are processed.

The baskets are then delivered to the pre-wash where they undergo a dual-cycle 110, 115 of a high-jet spray followed by a rinse in order to remove detritus from the surface of the needles. From here the baskets enter the treatment station whereby the pre-washed needles undergo an electro-polishing process 120 followed by a pre-treatment through immersion in a nitrite-acid bath 130 with a high pressure jet spray and blow-dry intermediate the treatment and post-treatment steps. Finally, the needles are removed from the nitric acid bath 130 and undergo a further high pressure jet spray and blow-dry before delivery to the cleaning station 95.

First, the needles undergo a pre-wash cycle in an ultrasonic bath 140 before delivery to an ultrasonic detergent bath 145 as a final clean of the needles before the rinse and drying stations. The needles may be subjected to an agitating action of the jig within the basket. The agitation has the effect of loosening the needles so as to fit any dislodged needles back into place, and more importantly, to shake loose water which may have adhered to the needles following the ultrasonic detergent wash.

The needles then go through a further ultrasonic rinse 155 followed by a hot water rinse 160 prior to a 3-step blow-dry phase 165 so to yield relatively dry bundles of needles. The heated needles, having a small amount of water adhered thereto, go through a 3-cycled oven dry prior to a final vacuum oven dry 175. Finally, the needles are delivered to the unloading zone 105 to be unloaded into the various packaging at the opposed end of the device 180.

In an alternative arrangement, the second ultrasonic rinse 160 may be replaced by a cold rinse, such as at 8-10° C. This has the effect of cooling the needles and therefore allowing shrinkage so as to provide greater gaps between the needles and so more effectively allow interstitial water between the needles to drain and a more efficient use of the blow-dry station 165 to remove moisture.

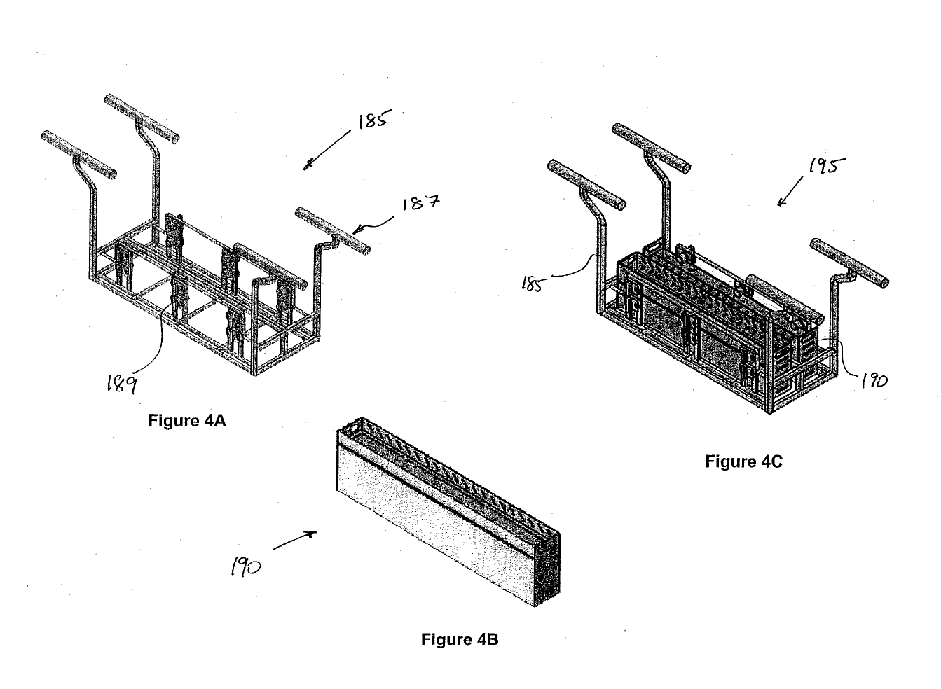

For the efficient transmission of the needles through the cleaning device according to the present invention, a basket 185 shown in FIG. 4A may include brackets 187 to engage the conveyor for the conveying of the jigs 190 into which the bundles of needles fit 195. The basket 185 may further include locking lugs 189 so as to securely engage the jigs as shown in FIG. 4C where 2 jigs are placed side by side and held in place by the engagement lugs 189.

The foregoing description is only exemplary of the principles of the invention. Many modifications and variations are possible in light of the above teachings. It is, therefore, to be understood that within the scope of the appended claims, the invention may be practiced otherwise than using the example embodiments which have been specifically described. For that reason the following claims should be studied to determine the true scope and content of this invention.

1. A system for cleaning a plurality of needles, the system comprising:
a conveyor for conveying a plurality of jigs through a series of stations, each of said plurality of jigs arranged to contain at least some of said needles;
said series of stations including:
a pre-treatment station for the-removal of detritus from said needles;
an electronic polishing station;
a post-treatment station for removal of material from a surface of said treated needles;
a cleaning station for cleaning said needles;
a drying station; and
an unloading station for unloading said needles from said plurality of jigs.

2. The system according to claim 1, wherein the pre-treatment station applies high pressure jet spray to said needles.

3. The system according to claim 1, wherein said plurality of jigs are mounted to baskets arranged to engage the conveyor.

4. The system according to claim 1, wherein said cleaning station is arranged to immerse said needles in an ultra-sonic bath.

5. The system according to claim 4, wherein said cleaning station is further arranged to immerse said needles in a detergent bath.

6. The system according claim 1, wherein the post-treatment includes immersion in nitric acid bath.

7. A method of cleaning a plurality of needles, the method comprising the steps of:
conveying a plurality of jigs through a series of stations, each of said plurality of jigs arranged to contain at least some of said needles;
pre-treating said needles for the removal of detritus;
electro-polishing the needles;
post-treating the needles to remove material from a surface of said treated needles;
cleaning said needles; and
drying said needles.

8. The method according to claim 7, wherein the step of drying comprises:
blow drying said needles; and
oven drying said needles.

9. A jig for containing a plurality of needles during a washing procedure, said jig comprising:
a containment into which said plurality of needles are placed; and
a cap placed on said containment for preventing floating of said plurality of needles.

10. The jig according to claim 9, wherein said cap and said containment are arranged so as to apply a known compression for a pre-determined number of plurality of needles within said jig.
